(19) United States
(12) Patent Application Publication
(10) Pub. No.: US 2013/0189320 A1
(43) Pub. Date: Jul. 25, 2013

(54) METHOD OF PREPARING A CONTROLLED RELEASE PARTICLE OF SOY ISOFLAVONE WITH BIODEGRADABLE POLYMER USING A SUPERCRITICAL FLUID EXTRACTION OF EMULSION (SFEE) PROCESS

(71) Applicant: Nano and Advanced Materials Institute Limited, Hong Kong (HK)

(72) Inventors: Kathy Qian LUO, Hong Kong (HK); Jinjie XU, Hong Kong (HK)

(73) Assignee: Nano and Advanced Materials Institute Limited, Hong Kong (HK)

(21) Appl. No.: 13/733,900

(22) Filed: Jan. 4, 2013

Related U.S. Application Data
(60) Provisional application No. 61/632,216, filed on Jan. 20, 2012.

(51) Int. Cl. A61K 9/14

(52) U.S. Cl. CPC A61K 9/14
USPC 424/400; 422/187; 514/456

(57) ABSTRACT

A method of preparing a controlled release particle of soy isoflavone (e.g. genistein) with a bio-degradable polymer is disclosed herein. The method employs a supercritical fluid extraction of emulsion (SFEE) process for encapsulating soy isoflavone into a bio-degradable polymer matrix (e.g. PLGA) to form a particle which is suitable for oral administration or inhalable administration in a controlled release manner and with an improved bioavailability of the soy isoflavone. A system for preparing the controlled release particle of the soy isoflavone with the bio-degradable polymer using the SFEE process is also disclosed herein.

### CROSS-REFERENCE TO RELATED APPLICATIONS

The present application claims priority from the U.S. provisional application Ser. No. 61/632,216 filed Jan. 20, 2012, the disclosure of which is incorporated herein by reference.

### FIELD OF THE INVENTION

The present invention relates to a method of preparing a controlled release particle of soy isoflavone (a subgroup of flavonoid characterized in soybeans or soybean products) with a bio-degradable polymer. In particular, the present invention relates to a method of preparing a solid form of soy isoflavone, e.g. genistein, and its derivatives, by using a supercritical fluid extraction of emulsion (SFEE) process for encapsulating soy isoflavone into a bio-degradable polymer matrix to form a controlled release particle suitable for oral administration or inhalable administration and with an improved bioavailability.

### TECHNICAL BACKGROUND

Oral route is the most common route of drug administration. The drug delivered by oral administration is usually in the form of powder, tablet or capsule, and is first dissolved in the gastrointestinal fluid along the GI tract and the dissolved drug subsequently permeates through the gastrointestinal membrane. However, oral route is not suitable for many drug molecules because of unacceptably low bioavailability caused by low water solubility, poor gastrointestinal membrane permeability, first pass metabolism, and instability in the gastrointestinal environment.

Soy isoflavones are phytoestrogens with chemical structures and physiological functions that are similar to those of the female hormone, estrogen. Thus, they can relieve estrogen-deficient diseases especially menopausal symptoms including hot flashes, osteoporosis and cardiovascular problems. To date, twelve main isoflavones have been characterized in soy bean or soy bean products including genistein, daidzein, and glycitein (aglycones), and their respective malonyl, acetyl, and glucosyl forms (glucosides) (Apers et al. 2004; Rostagno et al. 2004). Genistein has been widely used as healthcare products to relieve estrogen-deficient diseases especially menopausal symptoms but its therapeutic effects are hampered by its poor bioavailability. Two possible reasons for its low bioavailability are: its low water solubility and extensive first pass metabolism. It is found that incorporation into lipidic or polymer-based nanoparticles appears to remarkably help the oral delivery of flavonoids, as these particles can protect the drug from degradation in the gastrointestinal tract and also from first-pass metabolism in the liver (Leonarduzzi et al. 2010). Other researcher have tried various nanoapproaches including incorporation of genistein into topical nanoemulsion formulations composed of egg lecithin, medium chain triglycerides (MCT) or octyldodecanol (ODD) and water by spontaneous emulsification (Silva et al. 2009). Compared to the conventional methods of preparing polymer particles, a promising technique called supercritical fluid extraction of emulsions process (SFEE) shows its particular advantage which combines the flexibility of particle formulation using different emulsion systems with the efficiency of large-scale and continuous extraction with supercritical fluid. It was developed rapidly during the last five years and attracts a vast amount of attention (Chattopadhyay et al. 2006; Shekunov et al. 2006; Della Porta et al. 2008; Kluge et al. 2009; Kluge et al. 2009).

### SUMMARY OF THE INVENTION

The first object of the present invention is a method of preparing a controlled release particle of a soy isoflavone with a bio-degradable polymer. The method of the present invention includes using a supercritical fluid extraction of emulsion (SFEE) process to encapsulate a soy isoflavone into a bio-degradable polymer matrix to form a controlled release particle. The soy isoflavone that the method of the present invention is capable of encapsulating into a bio-degradable polymer matrix to form a controlled release particle includes genistein, daidzein, or glycitein (aglycones), or their respective malonyl, acetyl, or glucosyl forms (glucosides). The method of the present invention also includes preparing a double emulsion which contains an aqueous solution of a soy isoflavone (e.g. genistein) and an organic solution of the bio-degradable polymer prior to the SFEE process. The exemplary bio-degradable polymer of the present invention is poly(lactic-co-glycolic acid) (PLGA).

The second object of the present invention is a soy isoflavone-containing PLGA particle (e.g. genistein-containing PLGA particle) prepared by encapsulating genistein into a PLGA matrix using a supercritical fluid extraction of emulsion (SFEE) process as described herein. The resulting particle has a controlled drug release property and thereby improves the bioavailability of the encapsulated soy isoflavone.

### BRIEF DESCRIPTION OF THE DRAWINGS

FIG. 1 is a schematic diagram depicting the experimental setup of the supercritical fluid extraction of emulsion (SFEE) process.

FIG. 2 is SEM images of raw genistein (left panel) and the PLGA matrix encapsulating genistein after SFEE process (right panel).

FIG. 3 is a release profile of raw genistein versus encapsulated genistein.

### DEFINITIONS

As used herein, the term “supercritical fluid” refers to supercritical or near supercritical CO₂.

As used herein, the term “soy isoflavone” refers to the soy aglycone isoflavone, e.g., genistein, daidzein, and glycitein, etc., or their respective malonyl, acetyl, and glucosyl forms/soy glucoside isoflavone, e.g., daidzin, glycitin, genistin, acetyldaidzine, acetylglycitin, acetylgenistin, malonyldaidzin, malonylglycitin, and malonylgenistin, etc.

As used herein, the term “emulsion droplet” refers to water/oil/water emulsion droplet.

As used herein, the term “organic solvent” refers to dichloromethane.

### DETAILED DESCRIPTION OF THE INVENTION

In the following examples, poly(lactic-co-glycolic acid) (PLGA) is used as a bio-degradable polymer to encapsulate soy isoflavone so as to provide a controlled release system for the soy isoflavone when it is orally administered or through inhalation to a subject. Genistein is used as the soy isoflavone that is encapsulated into the bio-degradable polymer in the following examples. A genistein-containing PLGA particle is therefore prepared by the SFEE process as described herein. A double emulsion with the desired formulation is first prepared prior to the SFEE process. An example of how to prepare the double emulsion is described in Example 1. During the SFEE process, each emulsion droplet formed can be considered as a “miniature gas anti-solvent precipitator”, where supersaturation, particle nucleation, and particle growth occur after the removal of organic solvent. As a result, spherical shaped particles with small size can be obtained. An in vitro drug release experiment follows to verify the feasibility of protecting genistein by a polymer matrix. It should be noted that although PLGA and genistein are used as the bio-degradable polymer and the soy isoflavone respectively in the following examples, they are not intended to limit the scope of the present invention but simply for illustration purpose. It should also be understood that any suitable equivalents may be used to substitute the components/compounds/molecules as described in the following examples, provided that the technical effect after the substitution by the suitable equivalents according to the method of the present invention is substantially the same as described in the following examples, and/or the spirit and scope of the claims should not be departed due to the substitution.

The method of the present invention mainly employs a supercritical fluid extraction of emulsions (SFEE) process which aims to use a supercritical fluid to extract an organic solvent from the double emulsion in order to encapsulate the soy isoflavone (e.g. genistein) into the bio-degradable polymer matrix (e.g. PLGA matrix) to result in a controlled release particle after the extraction. The supercritical fluid used in the SFEE process is supercritical CO₂. The follow-up in vitro drug release study shows that the release of genistein from the genistein-containing PLGA particle is much slower than the raw/unprocessed genistein, which indicates that the genistein-containing PLGA particle is a promising system for long-term drug delivery. The study also shows the controlled release property of the encapsulated genistein based on the property of PLGA will lead to an improved bioavailability. Apart from genistein, the method of the present invention can be used to incorporate many other active pharmaceutical ingredients with any bio-degradable polymers to result in a controlled release system suitable for oral administration and inhalation with an improved bioavailability of the intended active ingredients.

### EXAMPLES

The present invention is now explained more specifically by referring to the following examples. These examples are given only for a better understanding of the present invention, and not intended to limit the scope of the invention in any way.

### Example 1

A water/oil/water (w/o/w) double emulsion is first prepared prior to the SFEE process as described herein. Ten (10) mg of genistein is dissolved in 1 mL of 0.1 M NaOH, and subsequently mixed with 10 mL dichloromethane which contains 200 mg PLGA using an ultrasonicator at 90 W for 1 min. The resulting w/o emulsion is then added into 1 wt % of PVA solution at a fixed organic to PVA aqueous phase ratio of 1:4. Another ultrasonication follows at 90 W for 1 min to form a w/o/w emulsion. An ice bath is used for cooling the emulsion during each ultrasonication.

### Example 2

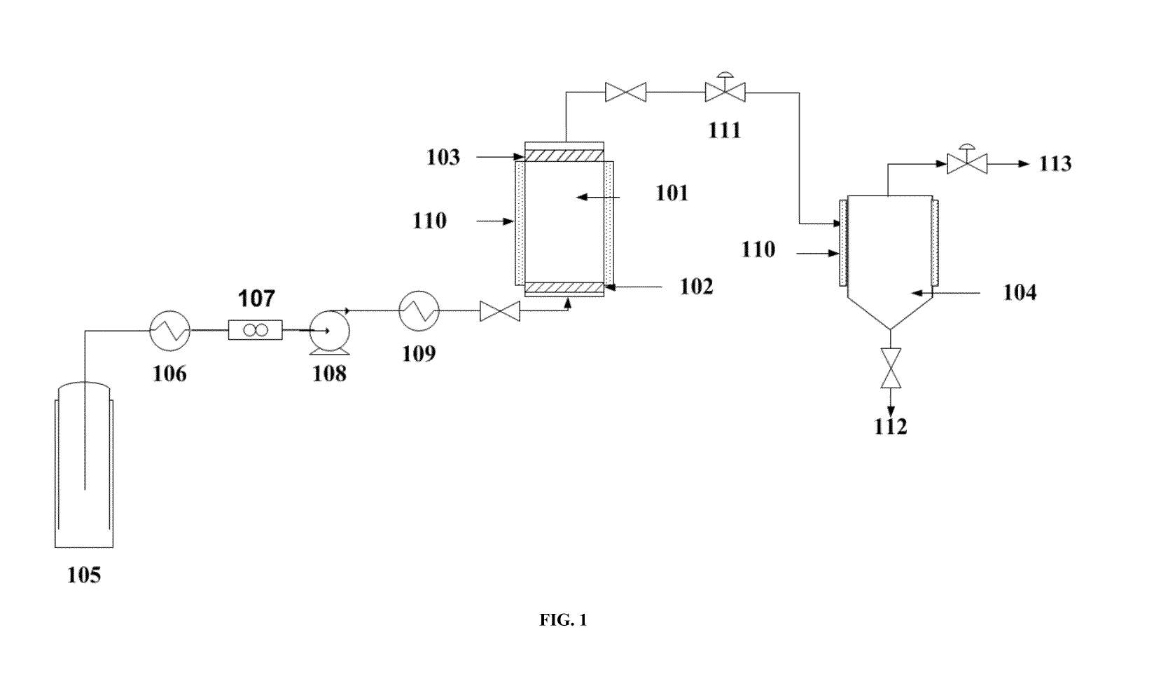

The experimental setup of SFEE process is illustrated in FIG. 1. The setup includes a precipitation chamber 101 in a volume of at least 400 mL. The precipitation chamber 101 is cylindrical and made of stainless steel in this example. The chamber 101 is also equipped with heating jacket 110 for keeping the precipitation chamber at certain temperature during the reaction between the supercritical fluid and the double emulsion. The prepared double emulsion from Example 1 is first loaded into the precipitation chamber 101. The supercritical CO₂ is created from a CO₂ module including a CO₂ tank 105 which is connected to a cooler 106 at one end. The cooler 106 includes a water/ethylene glycol circulating bath at −4° C. to maintain the CO₂ in the liquid phase prior to raising it to a desired temperature. The CO₂ module also includes a flow meter 107 which is connected to another end of the cooler 106 in order to monitor the CO₂ flow rate from the CO₂ tank 105 via the cooler 106. A high performance pump 108 is used to deliver the supercritical fluid of CO₂ after cooling to a heater 109 to heat up the fluid to the desired temperature before entering into the precipitation chamber 101. The supercritical CO₂ then enters at the bottom of the precipitation chamber 101 through a metal filter 102 having a pore size of 5 μm at a fixed flow rate. The filter 102 can improve the mass-transfer rate during the extraction of the organic solvent, i.e., dichloromethane. The double emulsion and the supercritical CO₂ react at the precipitation chamber 101 at the pressure above or near the supercritical point. The initial mass (in g) of the CO₂ used to produce the supercritical CO₂ is set at 40 times the volume (in mL) of dichloromethane used to prepare the double emulsion. The dichloromethane is extracted by supercritical CO₂ in the precipitation chamber 101 and recovered as a liquid in a low pressure cyclone separator 104. At the top of the chamber 101, there is another metal filter 103 which can prevent any resulted product escaping from the chamber 101 along with supercritical CO₂ stream. Before the gas effluent of dichloromethane and supercritical CO₂ enters into the cyclone separator 104, it passes through a back-pressure regulator 111 which is connected to the precipitation chamber 101 at one end and to the cyclone separator 104 at another end. The back-pressure regulator 111 is used to monitor and maintain the pressure of 101. A liquid state of dichloromethane is formed at the low pressure cyclone separator 104 surrounded by heating jacket 110 and being removed from the bottom thereof 112. Excess CO₂ will be removed from a vent 113 connected to the low pressure cyclone separator 104. The resulted suspension containing particles are collected from the bottom of the chamber 101 which is incorporated with the metal filter 102 and washed twice with distilled water by centrifugation at 20,000 rpm for 15 min. The particles collected from the chamber 101 are then re-suspended in pure water and subsequently freeze-dried for further analysis.

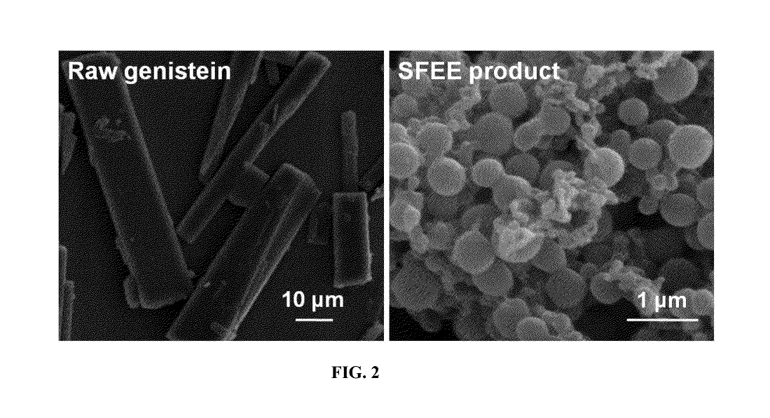

In a working example of preparing the controlled release particle of genistein with PLGA using the setup as illustrated in FIG. 1, the following conditions are used: CO₂ flow rate is 8 g/min; the pressure used in the precipitation chamber is about 80 bar; the precipitation temperature is about 35° C.; and the ratio of the supercritical CO₂ to dichloromethane is about 40 g/mL. FIG. 2 shows the morphology of the particle of genistein with PLGA prepared by the method of the present invention under a scanning electron microscope (SEM) (right panel) and compares with the particles prepared from raw genistein (left panel). It appears from the SEM images that the particle of genistein with PLGA has a nearly spherical shape and an average size of less than 1 μm. In comparison, the raw genistein is rectangular rod-shaped and the width (i.e., the shortest side of the rectangular rod-shaped raw genistein) is about 10 μm. PLGA matrix can protect genistein from degradation in the gastrointestinal tract and also from first-pass metabolism in the liver. Meanwhile, the nearly spherical shape of the genistein particle with the size less than 1 μm has a larger surface area to favor the interaction between the particle and the target cell in order to increase the chance of localizing on the target cell and delivering the genistein encapsulated in the particle in a controlled release manner.

### Example 3

A known weight of prepared particles from Example 2 is reconstituted in 1 mL of acetonitrile followed by moderate sonication to obtain a completely clear solution. Different samples are filtered using a 0.22 μm nylon syringe filter for HPLC analysis. The encapsulation efficiency is calculated as (Koushik et al. 2004):

$$\begin{matrix}Drug loading = \frac{the weight of encapsulated genistein}{the gross weight of the particles} & ( 1 ) \\ Encapsulation efficiency = \frac{actual genistein loading}{theoretical genistein loading} \times 100 \% & ( 2 )\end{matrix}$$

In the present invention, the theoretical drug loading is the amount of genistein to the amount of PLGA at the very beginning of the encapsulation experiments. Each experiment is carried out in duplicate. The encapsulation efficiency of genistein is calculated to be 87±0.9%. Such a high encapsulation efficiency may be attributed to the fact that the amount ratio of PLGA to genistein is relatively high at 20:1 and relatively low solubility of genistein in supercritical CO₂.

### Example 4

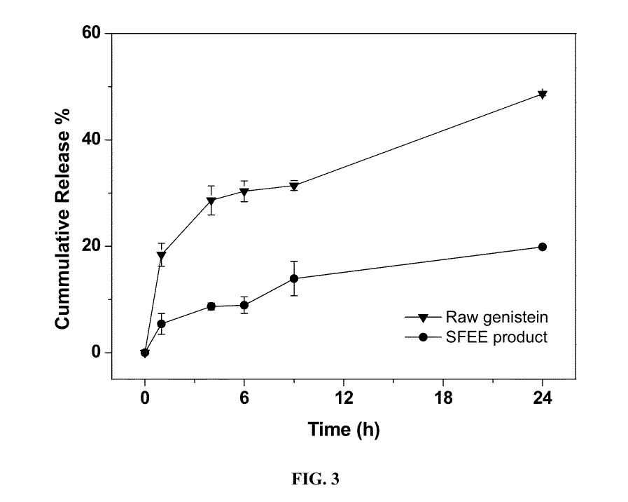

In the drug release study, an aqueous medium containing 1% Tween 80 is used to re-suspend the genistein-containing PLGA particles or the raw genistein. The cumulative release percentage of the encapsulated genistein from the PLGA coated particles and that of the raw genistein without PLGA for a 24-hour period is shown in FIG. 3. In the previously published reports, some of the drug loaded particle formulations displayed an initial high drug release (Birnbaum, 2000; Otsuka, 2002). That may be due to the presence of free and weakly bound drug on the surface of particulate carriers. In comparison, the genistein-containing PLGA particle of the present invention exhibits a much slower release profile of genistein with steadily increased release rate which indicates a homogeneous encapsulation of genistein by the PLGA polymers via the SFEE process of the present invention. In FIG. 3, the encapsulated genistein from the PLGA coated particles has a cumulative release percentage of below 20% within the first 24 hours while the raw genistein has over 40% of release over the 24-hour period. From this in vitro study, it could be said that the extended release profile of genistein-containing PLGA particles can increase the chance of genistein to be utilized in human body, which further leads to an improved bioavailability. The slower the release rate is, more available active ingredients are released to a specific site of action. The release profile of said encapsulated genistein in the PLGA matrix shows a nearly 2.4-fold reduction in the release rate as compared to the release rate of raw genistein in terms of the cumulative release percentage within 24 hours from T=0 (i.e., from the time of administering the same to a subject).

If desired, the different functions discussed herein may be performed in a different order and/or concurrently with each other. Furthermore, if desired, one or more of the above-described functions may be optional or may be combined.

Although various aspects of the invention are set out in the independent claims, other aspects of the invention comprise other combinations of features from the described embodiments and/or the dependent claims with the features of the independent claims, and not solely the combinations explicitly set out in the claims.

It is also noted herein that while the above describes exemplary embodiments of the invention, these descriptions should not be viewed in a limiting sense. Rather, there are several variations and modifications which may be made without departing from the scope of the present invention as defined in the appended claims.

What we claim:

1. A method of preparing a controlled release particle of soy isoflavone with a bio-degradable polymer for oral administration or inhalable administration to a subject, said method comprising employing a supercritical fluid to extract an organic solvent from a double emulsion containing an aqueous solution of said soy isoflavone and an organic solution of said bio-degradable polymer in order to form a controlled release particle after the extraction of said organic solvent.

2. The method of claim 1, wherein said supercritical fluid is supercritical or near supercritical CO₂.

3. The method of claim 1, wherein the initial mass of CO₂ used to produce said supercritical fluid is 40 times the initial volume of said organic solvent used to produce said double emulsion.

4. The method of claim 1, wherein said controlled release particle of soy isoflavone is in a powder form.

5. The method of claim 1, wherein said biodegradable polymer is poly(lactic-co-glycolic acid) (PLGA).

6. The method of claim 1, wherein said organic solvent is dichloromethane.

7. The method of claim 1 further comprises preparing a double emulsion prior to said employing the supercritical fluid, wherein said double emulsion is prepared by:
a. dissolving 10 mg of genistein in 1 mL of 0.1 M NaOH to form the soy isoflavone aqueous solution;
b. mixing the soy isoflavone aqueous solution with 10 mL of dichloromethane which contains 200 mg of poly(lactic-co-glycolic acid) by a first ultrasonication at 90 W for 1 minute to form a first emulsion; and
c. adding the first emulsion into 1 wt % of PVA solution at a ratio of 1:4 followed by a second ultrasonication at 90 W for 1 min to obtain said double emission.

8. The method of claim 1, wherein said employing the supercritical fluid comprises:
a. loading said double emulsion into a precipitation chamber;
b. producing the supercritical fluid by a CO₂ module;
c. passing said supercritical fluid from said CO₂ module to the bottom of said precipitation chamber through a metal filter at a fixed flow rate;
d. reacting said supercritical fluid with the double emulsion in said precipitation chamber at a pressure above or near the supercritical point;
e. extracting the organic solvent from said double emulsion by said supercritical fluid to a low pressure cyclone separator in where said organic solvent is recovered as a liquid;
f obtaining a suspension containing particles from the bottom of said precipitation chamber followed by washing said particles twice with distilled water through centrifugation at 20,000 rpm for 15 min; and
g. re-suspending the particles after centrifugation in pure water followed by freeze-drying the suspension containing the particles for storage or for future use.

9. The method of claim 8, wherein said bottom of the precipitation chamber comprises a metal filter having a pore size of 5 μm which is configured to improve the mass-transfer rate during the extraction of the organic solvent.

10. The method of claim 1, wherein said soy isoflavone is soy aglycone isoflavone or soy glucoside isoflavone selected from a group consisting of genistein, daidzein, glycitein, daidzin, glycitin, genistin, acetyldaidzin, acetylglycitin acetylgenistin malonyldaidzine, malonylglycitin, and malonylgenistin.

11. A composition comprising a plurality of the controlled release particles of soy isoflavone with said bio-degradable polymer prepared by the method of claim 1 for oral administration or inhalable administration to a subject in a controlled release manner.

12. The composition of claim 11, wherein the genistein-containing PLGA particles have an encapsulation efficiency of about 87%.

13. The composition of claim 11, wherein the controlled release particles of soy isoflavone with said bio-degradable polymer has about 2.4-fold reduction in drug release rate as compared to that of raw soy isoflavone within 24 hours after administration to a subject in needs thereof.

14. A controlled release particle of soy isoflavone prepared by the method of claim 1 has an average particle size of less than 1 μm and is in a nearly spherical shape.

15. A system for preparing a controlled release particle of soy isoflavone with a bio-degradable polymer for oral administration comprising a precipitation chamber, a CO₂ module and a low pressure cyclone separator,
wherein said precipitation chamber is configured to carry a double emulsion for reaction with a supercritical fluid to take place; said CO₂ module is configured to produce the supercritical fluid for said reaction to take place in said precipitation chamber; and said low pressure cyclone separator is configured to remove said organic solvent from said double emulsion after depressurization.

16. The system of claim 15, wherein said precipitation chamber is cylindrical, made of stainless steel and at least 400 mL in volume.

17. The system of claim 15, wherein said CO₂ module comprises a CO₂ tank, a cooler, a flow meter, a high performance CO₂ pump and a heater, wherein said CO₂ tank is connected to said cooler at one end, said flow meter is connected to said cooler at another end to monitor the flow rate of CO₂ from said CO₂ tank via said cooler to said heater.

18. The system of claim 15 further comprises a back-pressure regulator which is connected to said precipitation chamber at one end and to said low pressure cyclone separator at another end, wherein said low pressure cyclone separator is configured to recover the gaseous state of said organic solvent after reaction in said precipitation chamber into liquid state under depressurization such that said organic solvent as a liquid is removable from the bottom of said low pressure cyclone separator.

19. The system of claim 15, wherein said precipitation chamber further comprises at least one metal filter which is situated at the bottom of said precipitation chamber for improving the mass-transfer rate of said organic solvent during the extraction.

20. The system of claim 17, wherein said cooler comprises a water/ethylene glycol circulating bath at a low temperature to maintain the CO₂ in the liquid phase prior to raising the CO₂ flow to a desired temperature in said heater.
